(19) United States
(12) Patent Application Publication
(10) Pub. No.: US 2021/0259998 A1
(43) Pub. Date: Aug. 26, 2021

(54) COMBINATION MEDICATION AND METHOD OF ADMINISTRATION

(71) Applicant: Sharai Amaya, Greenwood, SC (US)

(72) Inventor: Sharai Amaya, Greenwood, SC (US)

(21) Appl. No.: 16/855,388

(22) Filed: Apr. 22, 2020

Related U.S. Application Data
(60) Provisional application No. 62/980,328, filed on Feb. 23, 2020.

(51) Int. Cl. A61K 31/167; A61K 31/5415; A61K 31/19

(52) U.S. Cl. CPC A61K 31/167; A61K 31/19; A61K 31/5415

(57) ABSTRACT

A pharmaceutical formulation for the treatment of pain and method of administration. The pharmaceutical formulation comprises a therapeutically effective dosage of acetaminophen and a therapeutically effective dosage of a non-steroidal anti-inflammatory drug (NSAID) combined in a single unit dosage form. The pharmaceutical formulation is orally administered. A method of treating pain by orally administering the pharmaceutical formulation to a human is also provided.

### CROSS-REFERENCE

This application claims priority from Provisional Patent Application Ser. No. 62/980,328 filed on Feb. 23, 2020.

### BACKGROUND

Pain is a generalized term that describes discomfort somewhere in a person's body. Pain is relative and ranges from minor distress to debilitating suffering. Pain can be constant or can only occur during a certain condition, such as the movement of an injured body part. Pain can also be acute, such as immediately following an injury, or can be chronic due to a long term injury or condition. Just as pain is unique to an individual, pain treatments are also individualized. One person may not require any intervention for an injury, whereas another may require narcotics to control similar pain.

Non-steroidal anti-inflammatory drugs (NSAIDs) are commonly used as first-line medications for the treatment of pain, fever, and inflammation. However, as previously mentioned, pain type severity and pain relief tend to be extremely individualized. NSAIDs block the production of certain body chemicals, such as prostaglandins that cause inflammation and associated pain. NSAIDs are good at treating pain caused by slow tissue damage. Acetaminophen is another commonly used first-line medication for the treatment of pain, but it is a different class of pain relieving agent. Acetaminophen is not an anti-inflammatory agent and eases pain through a different mechanism.

Often, more than one different medication may be employed in an attempt to relieve the pain using different mechanisms. Acetaminophen and an NSAIDs, such as ibuprofen, are commonly prescribed together by a physician or other advanced health care practitioner to combat pain or other medical symptoms. Consuming both medications currently requires taking multiple pills of two separate medications or swallowing two different types of liquids. Individuals may have difficulties in determining and taking the proper amount of each medication. Without a combined dosage of the two drugs, patients may inadvertently confuse the dosage of each drug, thereby becoming noncompliant with their prescribed pain management regimen.

Accordingly, there is a great need for a unique pharmaceutical composition constructed to facilitate pain relief. With the opioid crisis in the United States reaching epidemic proportions, this invention offers a simple, non-narcotic alternative. The present invention combines two different non-narcotic pain relieving medications into a single delivery formulation. The pharmaceutical composition enables users to easily combine and consume both medications simultaneously in the correct dosage in accordance with their involved physician's recommendations. The invention advantageously reduces the number of pills required to optimize pain relief for a wide variety of applications. The pharmaceutical composition is adaptable for both adult and pediatric patients.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one aspect thereof, comprises a pharmaceutical formulation for use in the treatment of pain. The pharmaceutical formulation is orally administered in a single unit dosage form. The single unit dosage form comprises a pill, a tablet, a chewable, a capsule, dissolving powder, dissolving film, a sachet, or a liquid. The pharmaceutical formulation may comprise an enteric coating.

The pharmaceutical formulation comprises a therapeutically effective dosage of acetaminophen or a pharmaceutically acceptable salt thereof. The therapeutically effective dosage of acetaminophen typically ranges from 160 mg for a pediatric dosage up to 500 mg for an adult dosage. The pharmaceutical formulation further comprises therapeutically effective dosage of a non-steroidal anti-inflammatory drug (NSAID) or a pharmaceutically acceptable salt thereof combined with the therapeutically effective dosage of acetaminophen. The therapeutically effective dosage of the NSAID typically ranges from 100 mg for a pediatric dosage up to 400 mg for an adult dosage. The NSAID comprises at least one of a propionic acid derivative, an acetic acid derivative, a biphenylcarboxylic acid derivative, an oxicam, and pharmaceutically acceptable salts thereof.

An additional embodiment of the present invention comprises a method of treating pain in a human. The method comprises orally administering a therapeutically effective dosage of acetaminophen and a therapeutically effective dosage of a NSAID combined in a single unit dosage form to the human. The single unit dosage form comprises a pill, a tablet, a chewable, a capsule, or a liquid. The therapeutically effective dosage of acetaminophen typically ranges from 160 mg for a pediatric dosage up to 500 mg for an adult dosage. The therapeutically effective dosage of the NSAID will vary depending on the specific NSAID chosen. For example, the therapeutically effective dosage of ibuprofen typically ranges from 100 mg for a pediatric dosage up to 400 mg for an adult dosage. The NSAID comprises at least one of a propionic acid derivative, an acetic acid derivative, a biphenylcarboxylic acid derivative, an oxicam, and pharmaceutically acceptable salts thereof.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### DETAILED DESCRIPTION OF THE INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

The present invention discloses a unique pharmaceutical composition of acetaminophen combined with a NSAID to facilitate pain relief. The pharmaceutical composition may be delivered as a pill, a tablet, a capsule, a chewable tablet, a dissolving powder sachet, or dissolving film or a liquid for simple consumption. The present invention enables users to easily take the correct dosage of two different medications simultaneously in one unit thereby ensuring the optimal combination and dosage of the acetaminophen and the NSAID to combat aches and pains. The pharmaceutical composition is particularly effective for post-operative patients and decreases the likelihood of narcotic usage to control the pain. The pharmaceutical composition is similarly effective for treating pain caused by headaches, arthritis, inflammation, dental pain, and the like.

The term “therapeutically effective dose” as used herein is meant to refer to an amount of a composition or compound effective to result in the amelioration of symptoms associated with a condition, or to provide a beneficial therapeutic effect, such as, but not limited to, at least partial pain relief, and/or a reduction in inflammation.

The term “single unit dosage form” as used herein is meant to refer to the co-administration of at least two compounds combined in a single delivery form, such as, but not limited to, a pill, a capsule, a tablet, a chewable, a liquid, and the like.

The term “NSAID” as used herein represents members an analgesic drug class that reduces pain, fever, and inflammation. NSAIDs as used herein include any agent with these effects as well as any pharmaceutically acceptable salt thereof.

The term “pharmaceutically acceptable salt” as used herein is meant to refer to those salts of biological compounds which retain the biological effectiveness and properties of the free compound (i.e. free bases and/or acids), and can include, but are not limited to, pharmaceutically acceptable acid and/or base addition salts, as well as pharmaceutically acceptable cationic and/or anionic salts. Examples of pharmaceutically acceptable salts include, for example, acid addition salts, such as hydrochloride salts, alkali metal salts, such as, sodium and potassium, alkaline earth salts, ammonium salts, and the like. It should also be understood that the compounds and/or pharmaceutically acceptable salts thereof as described herein may be provided in their hydrate and/or solvate forms.

One embodiment of the present invention comprises a pharmaceutical formulation for the treatment of pain formulated in a single unit dosage form. The pharmaceutical formulation comprises a combination of acetaminophen and a NSAID. The single unit dosage form of the combination of the acetaminophen and the NSAID may be an orally administrable pill, a tablet, a capsule, a chewable, a liquid or liquid suspension, a dissolving powder, a sachet, an orally dissolving film, or any other form of an oral deliverable medication.

The single unit dosage form of the pharmaceutical formulation allows the patient to simultaneously take a proper dose of the acetaminophen and a NSAID. This particular pharmaceutical formulation is advantageous as it combines two commonly separately prescribed medications for pain relief as an effective alternative to narcotic medications in a single delivery mechanism. The combination of acetaminophen and a NSAID has a synergistic effect on the treatment of pain as the different medications ease pain through different mechanisms.

The pharmaceutical formulation may contain one or more binders with adhesive properties that serve to promote cohesiveness in the solid dosage form. The formulation may contain one or more disintegrants to promote acceptable disintegration rate in the gastrointestinal tract. “Disintegrant” means a material in the formulation that acts to cause the solid matrix to break up when the solid dosage form comes in contact with aqueous media. The formulation may contain lubricants to prevent pharmaceutical ingredients from clumping together during manufacturing. The formulation may contain excipients, meaning materials other than the two active pharmaceutical ingredients (acetaminophen and NSAID), such as flavorants, preservatives, and dilutants. Excipient ingredients may be used in making a film coating.

The pharmaceutical formulation comprises a therapeutically effective dosage of acetaminophen or a pharmaceutically acceptable salt thereof; and a therapeutically effective dosage of a NSAID or a pharmaceutically acceptable salt thereof. The therapeutic effect is the reduction or relief of pain. The pharmaceutical formulation may further comprise an enteric coating to permit transit of the pharmaceutical formulation through the stomach to the small intestine before the medications are released.

Examples of suitable NSAIDs include aspirin, propionic acid derivatives, acetic acid derivatives, fenamic acid derivatives, biphenylcarboxylic acid derivatives, oxicams, and pharmaceutically acceptable salts thereof. Examples of propionic acid derivatives include ibuprofen, naproxen, flurbiprofen, fenbufen, fenoprofen, indoprofen, ketoprofen, fluprofen, piprofen, carprofen, oxaprozin, pranoprofen, suprofen, and the like. Examples of acetic acid derivatives include indomethacin, diclofenac, sulindac, tolmetin, and the like. Examples of fenamic acid derivatives include mefenamic acid, meclofenamic acid, flufenamic acid, and the like. Examples of biphenylcarboxylic acid derivatives include diflunisal, flufenisal, and the like. Examples of oxicams include piroxicam, sudoxicam, isoxicam, meloxicam, and the like. Therefore, the NSAID will comprise at least one of these medications.

The therapeutically effective dose of acetaminophen typically ranges from 160 mg to 500 mg but may be higher or lower, such as 100 mg-580 mg or higher, depending on the specific need of a patient. A 160 mg dose is typical for a pediatric patient, and a 500 mg dose is typical for an adult patient. A preferred NSAID is ibuprofen. The therapeutically effective dose of ibuprofen typically ranges from 100 mg to 400 mg but may be higher or lower, such as 50 mg-850 mg or higher, depending on the specific need of a patient. A 100 mg dose is typical for a pediatric patient, and a 400 mg dose is typical for an adult patient.

Other preferred NSAIDs include naproxen sodium, meloxicam, diclofenac, ketorolac, and indomethacin. The therapeutically effective dose of naproxen sodium typically ranges from 220 mg-550 mg but may be higher or lower depending on the specific needs of the patient. The therapeutically effective dose of naproxen sodium typically ranges from 220 mg-550 mg but may be higher or lower depending on the specific needs of the patient. The therapeutically effective dose of meloxicam typically ranges from 5 mg-15 mg but may be higher or lower depending on the specific needs of the patient. The therapeutically effective dose of diclofenac typically ranges from 25 mg-50 mg but may be higher or lower depending on the specific needs of the patient. The therapeutically effective dose of ketorolac typically ranges from 5 mg-30 mg but may be higher or lower depending on the specific needs of the patient. The therapeutically effective dose of indomethacin typically ranges from 25 mg-50 mg but may be higher or lower depending on the specific needs of the patient.

The following are nonlimiting examples provided to further illustrate formulations of the present invention.

### EXAMPLES

### Formulation 1

A tablet of the pharmaceutical formulation is prepared with the following ingredients and dosages:

| Ingredient | mg |
|---|---|
| Acetaminophen | 500 |
| Ibuprofen | 400 |

This formulation represents a typical adult preparation of the pharmaceutical formulation for the treatment of pain. The formulation is repeatable every 4-6 hours as necessary up to a maximum of 3000 my of acetaminophen in a 24 hour period.

### Formulation 2

A tablet of the pharmaceutical formulation is prepared with the following ingredients and dosages:

| Ingredient | mg |
|---|---|
| Acetaminophen | 575 |
| Naproxen | 440 |

This formulation represents a typical adult preparation of the pharmaceutical formulation for the treatment of pain.

### Formulation 3

A tablet of the pharmaceutical formulation is prepared with the following ingredients and dosages:

| Ingredient | mg |
|---|---|
| Acetaminophen | 550 |
| Meloxicam | 15 |

This formulation represents a typical adult preparation of the pharmaceutical formulation for the treatment of pain.

### Formulation 4

A tablet of the pharmaceutical formulation is prepared with the following ingredients and dosages:

| Ingredient | mg |
|---|---|
| Acetaminophen | 160 |
| Ibuprofen | 100 |

This formulation represents a typical pediatric preparation of the pharmaceutical formulation for the treatment of pain.

An additional embodiment of the present invention comprises a method for treating pain in a human. The method comprises orally administering a administering a therapeutically effective dosage of acetaminophen and a therapeutically effective dosage of a NSAID combined in a single unit dosage form to the human. The therapeutically effective dose of acetaminophen typically ranges from 160 mg to 500 mg but may be higher or lower depending on the specific need of a patient. The NSAID may include propionic acid derivatives, acetic acid derivatives, fenamic acid derivatives, biphenylcarboxylic acid derivatives, oxicams, and pharmaceutically acceptable salts thereof.

A preferred NSAID is ibuprofen. The therapeutically effective dose of ibuprofen typically ranges from 100 mg to 400 mg but may be higher or lower depending on the specific need of a patient. The 100 mg dose is typical for a pediatric patient, and the 400 mg dose is typical for an adult patient, but the dosages may vary depending on the patient's weight. The single unit dosage form of the combination of the acetaminophen and the NSAID may be an orally administrable pill, a tablet, a capsule, a chewable, a dissolving powder, a dissolving film, a dissolving tablet, a sachet, a liquid, or any other form of an oral deliverable medication. The dosage is repeatable. For example, one single unit dosage taken every 4-6 hours, or two single unit dosages taken every 8 hours. A maximum dose of acetaminophen is typically 3000 mg in any 24 hour period.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A pharmaceutical formulation in a single unit dosage form comprising:
a therapeutically effective dosage of acetaminophen; and
a therapeutically effective dosage of a non-steroidal anti-inflammatory drug (NSAID).

2. The pharmaceutical formulation of claim 1, wherein the therapeutic effect is pain relief.

3. The pharmaceutical formulation of claim 1, wherein the single unit dosage form is a pill, a tablet, or a capsule.

4. The pharmaceutical formulation of claim 1, wherein the single unit dosage form is a liquid.

5. The pharmaceutical formulation of claim 1, wherein the NSAID is a propionic acid derivative.

6. The pharmaceutical formulation of claim 1, wherein the NSAID is ibuprofen or a pharmaceutically acceptable salt thereof.

7. The pharmaceutical formulation of claim 1, wherein the NSAID is an acetic acid derivative.

8. The pharmaceutical formulation of claim 1, wherein the NSAID is a biphenylcarboxylic acid derivative.

9. The pharmaceutical formulation of claim 1, wherein the NSAID is an oxicam.

10. The pharmaceutical formulation of claim 1, wherein the therapeutically effective dosage of acetaminophen ranges from 160 mg to 500 mg.

11. The pharmaceutical formulation of claim 1, wherein the therapeutically effective dosage of the NSAID ranges from 100 mg to 400 mg.

12. A pharmaceutical formulation for the treatment of pain comprising:
a therapeutically effective dosage of acetaminophen or a pharmaceutically acceptable salt thereof;
a therapeutically effective dosage of a non-steroidal anti-inflammatory drug (NSAID) or a pharmaceutically acceptable salt thereof and wherein the pharmaceutical formulation is orally administered in a single unit dosage form.

13. The pharmaceutical formulation of claim 12, wherein the single unit dosage form is a pill, a tablet, a capsule, a liquid, or a chewable.

14. The pharmaceutical formulation of claim 12, wherein pharmaceutical formulation further comprises an enteric coating.

15. The pharmaceutical formulation of claim 12, wherein the NSAID comprises at least one of a propionic acid derivative, an acetic acid derivative, a fenamic acid derivative, a biphenylcarboxylic acid derivative, an oxicam, and pharmaceutically acceptable salts thereof.

16. The pharmaceutical formulation of claim 12, wherein the therapeutically effective dosage of acetaminophen ranges from 160 mg to 500 mg, and the therapeutically effective dosage of the NSAID ranges from 100 mg to 400 mg.

17. A method of treating pain in a human, comprising:
orally administering a therapeutically effective dosage of acetaminophen and a therapeutically effective dosage of a non-steroidal anti-inflammatory drug (NSAID) combined in a single unit dosage form to the human.

18. The method of claim 17, wherein the therapeutically effective dosage of acetaminophen ranges from 160 mg to 500 mg, and the therapeutically effective dosage of the NSAID ranges from 100 mg to 400 mg.

19. The method of claim 17, wherein the single unit dosage form is a pill, a tablet, a capsule, a liquid, or a chewable.

20. The method of claim 17, wherein the NSAID is a propionic acid derivative.
